(19) United States
(12) Patent Application Publication
(10) Pub. No.: US 2026/0334894 A1
(43) Pub. Date: Oct. 29, 2026

(54) WHEELCHAIR, ESPECIALLY MEDICAL WHEELCHAIR

(71) Applicant: EOS IMAGING, PARIS (FR)

(72) Inventors: Abderrahim EDDAOUDI, FRANCONVILLE (FR); Stéphane BLEAS, PARIS (FR)

(73) Assignee: EOS IMAGING, PARIS (FR)

(21) Appl. No.: 19/118,380

(22) PCT Filed: Oct. 6, 2022
(86) PCT No.: PCT/EP2022/077805

§ 371 Date: Apr. 4, 2025

(51) Int. Cl. A61B 6/04; A61B 6/10; A61G 5/10

(52) U.S. Cl. CPC A61B 6/0478; A61B 6/102; A61G 5/1035; A61G 2210/50

(57) ABSTRACT

A wheelchair including a seat, first wheels, and second wheels higher than the first wheels. The first wheels are retractable so as to be higher than the second wheels, when retracted.

### FIELD

The invention relates to the technical field of wheelchairs, and preferably more specifically to the technical field of medical wheelchairs.

### BACKGROUND

According to a first prior art, there exists a wheelchair with a seat on which a person can sit down and which is supported by rolling wheels so that the siting person can drive on the floor. The sitting person can be a permanently disabled or a temporary disabled person or a non-ambulatory patient within a hospital for instance. This is relatively easy to move on when the ground is flat. However, when there are some obstacles and/or some relief variations, like for instance a step to climb, this may become very burdensome to overcome for the sitting person. Not only shall he or she often receive the help of another walking person, but also this help may request a notable effort even from the helping walking person, especially when the sitting person is heavy, although the step to climb may not be that high.

Of course, a system may be implemented which would help the sitting person to climb the step, while quietly staying seated in the wheelchair, with or without the help of a walking person. However, this system would probably be complex and cumbersome, although it might be efficient.

According to a second prior art, for instance disclosed in prior art US 20160346144, it is known a patient chair with an adjustable height for radiology imaging. However, this second prior art is of little use when there is a step to climb with the wheelchair having a person already sitting in it.

Considering the preferred application of medical wheelchairs, a radiological apparatus may have a motorized platform, which allows for a positioning of the (valid, not disabled) patient in standing position at several different heights, preferably at two different heights:
A first exam height, at for instance 10 cm from the floor, which allows the patient to climb on the platform, but lets the feet out of the X-ray field,
A second exam height, for instance 43 cm from the floor, which allows for the X-ray tube to go below the patient's feet, and allows the patient to get full body scans (from his feet's soles to the top of the head).

Often, this platform cannot be removed from the system, implying a physical step to climb of a notable height, for instance of a minimum of 10 cm.

For non-ambulatory patients, the patient needs to be imaged in a radiolucent wheelchair. Transferring the patient from his personal wheelchair to the radiolucent wheelchair can be difficult, depending on his weight and handicap. It is best to do it in an open environment, with access to all sides of both chairs. Therefore, it is better to perform the transfer outside of the acquisition zone of the radiological apparatus.

The radiolucent wheelchair with the patient in it has then to be placed in the acquisition zone of the radiological system, preferably an X-ray system, on the platform, to perform the patient imaging acquisition exam.

Therefore, the wheelchair should allow the climbing of this 10 cm step with the patient sitting on it, safely, and without excessive force from the helping operator. It should also allow to climb down from the platform after the exam in the same comfortable conditions.

### SUMMARY

The object of the present invention is to alleviate at least partly the above mentioned drawbacks.

More particularly, the invention is looking for a system which would be altogether rather simple and easy, while being efficient by allowing the sitting person to remain quietly on the wheelchair, while a helping walking person would need little effort to help the wheeling chair carrying the sitting person to successfully climb the step. The preferred context which the invention deals with corresponds to a situation where the height of the step to climb is known in advance and remains constant, like for instance a predetermined step height within a radiological apparatus.

Therefore, the invention proposes to have second wheels at the right height, that is at the height of the step to climb, while first wheels on the ground are retractable, so that when the second wheels have taken over the first wheels by bearing on the step to climb, the first wheels are retracted so as let the wheelchair roll on the step without having anymore the first wheels rolling on the ground, thereby allowing for the wheelchair bearing the sitting person to smoothly go from the ground to a higher step to climb while roughly keeping the seat, and the sitting person in it, at the same altitude during the transfer from a first lower height of the ground onto a second higher height of the step to climb.

This first object of the invention is achieved with a wheelchair comprising: a seat, first wheels, wherein it also comprises: second wheels higher than the first wheels, and wherein: the first wheels are retractable so as to be higher than the second wheels, when retracted.

The second wheels are higher than the first wheels, with respect to the ground on which the wheelchair is located and on which the wheelchair may roll. The lowest part of the periphery of the second wheels, which could roll on the ground, is higher than the lowest part of the periphery of the first wheels, which therefore will roll on the ground. Preferably, the rotation axis of the second wheels are higher than the rotation axis of the first wheels too.

The first wheels are retractable so as to be higher than the second wheels when retracted, with respect to the ground on which the wheelchair is located and on which the wheelchair may roll. The lowest part of the periphery of the first wheels, which could roll on the ground, is higher than the lowest part of the periphery of the second wheels, which therefore will roll on the ground. Preferably, the rotation axis of the first wheels are higher than the rotation axis of the second wheels too.

This first object of the invention is also achieved with a radiological system, wherein it comprises: a wheelchair according to any of preceding claims, and a radiological apparatus comprising: a gantry encapsulated within a cover, a patient base plate, 2 radiation sources with imaging directions orthogonal to each other, sliding vertically so as to perform vertical scanning of a patient standing on said base plate, said gantry cover top view being L shaped, each of said 2 radiation sources: being located: outside said L shaped gantry cover, inside angular sector of said L, and being encapsulated within a cover sliding vertically with said radiation source it encapsulates.

Such a radiological system and such a radiological apparatus are preferably as the ones claimed and described in patent application WO 2021090042 which is hereby incorporated by reference.

When the radiology apparatus is used by a disabled patient or by a non-ambulatory patient, the base plate in this radiology apparatus is a step to climb by this disabled patient or by this non-ambulatory patient sitting in a wheelchair. If this disabled patient or this non-ambulatory patient sits in a wheelchair according to embodiments of the invention, he will be able to undergo a medical examination within the radiological apparatus with a maximized comfort, whereas if he would sit in a prior art wheelchair, he would encounter discomfort during installation before performance of this medical examination. Therefore, this makes an invaluable asset of the wheelchair according to the invention, when used within a radiological apparatus having a patient base plate or a patient elevating platform.

Some hospitals may even not be willing to buy the radiological apparatus, if not associated with a wheelchair according to the invention, into a radiological system combining the wheelchair according to the invention with a radiological apparatus presenting a patient base plate or elevating platform not at the same altitude than the rest of the floor. Some hospitals would consider as globally deficient a radiological system which would work perfectly with walking patients and which would provide for much discomfort to disabled or non-ambulatory patients.

According to embodiments of the invention, it aims at proposing a radio-transparent chair for adults and children with a mechanism allowing it to place the patient in the chair on a platform which is notably higher than the floor, for instance on a 10 cm high platform.

According to embodiments of the invention, the wheelchair is a device allowing for the acquisition of frontal and lateral images for non-ambulatory patients in functional sitting position. Non-ambulatory patients are patients unable to walk without assistance of another person, including wheelchair users and patients having trouble staying still while standing without assistive device. Being able to image these patients will allow them to benefit of radiological imaging low dose exams, 3D modelling and patient-specific parameters as well as, if necessary, spine module for the pre-operative spine surgical planning, even when the corresponding radiological apparatus presents a patient platform higher than the floor ground. Patients imaged in this wheelchair will be given the opportunity to benefit the full care pathway offered by a radiological apparatus presenting a patient platform higher than the floor ground.

Preferred embodiments comprise one or more of the following features, which can be taken separately or together, either in partial combination or in full combination, with this first object of the invention.

Preferably, the wheelchair also comprises: 2 first front wheels associated to 2 second front wheels, 2 first rear wheels associated to 2 second rear wheels.

Hence, the smooth transfer from the first lower height of the ground level to the second higher height (second higher height being higher than first lower height) of the step level can be performed step by step, either first front part of the wheelchair and second rear part of the wheelchair, or first rear part of the wheelchair and second front part of the wheelchair.

Preferably, the wheelchair also comprises: a first front locking mechanism securing the positions of the first front wheels when not retracted, a first rear locking mechanism securing the positions of the first rear wheels when not retracted.

There is preferably at least a first locking mechanism securing the positions of at least part of the first wheels when not retracted.

Hence, when the wheelchair is driven on a given level at the same height, risk is reduced or cancelled, that the first wheels retract when not wanted, for instance when touching a small obstacle or when encountering small relief variations, thereby unbalancing the wheelchair, what is already quite uncomfortable for the sitting person, but thereby also risking a fall on the ground of the sitting person, what becomes quite dangerous.

Preferably, the wheelchair also comprises: a second front locking mechanism securing the locking of the first front locking mechanism, a second rear locking mechanism securing the locking of the first rear locking mechanism.

There is preferably at least a second locking mechanism securing the locking of the first locking mechanism or of at least one of the first locking mechanisms.

Hence, thanks to this double cascading locking system, the locking of the retractable first wheels can practically no more be activated by accident without wanting to do so, even by an absent-minded helping walking person.

Preferably the wheelchair also comprises one third front wheel associated to said two first front wheels and to said two second front wheels, one third rear wheel associated to said two first rear wheels and to said two second rear wheels, unlocking of the second front locking mechanism is automatically and mechanically driven by pressure on the third front wheel being above a given threshold corresponding to the wheelchair loaded on the second front wheels rather than on the first front wheels, this pressure above a given threshold automatically and mechanically triggering a lock visual indicator showing that the second front locking mechanism is unlocked, unlocking of the second rear locking mechanism is automatically and mechanically driven by pressure on the third rear wheel being above a given threshold corresponding to the wheelchair loaded on the second rear wheels rather than on the first rear wheels, this pressure above a given threshold automatically and mechanically triggering a lock visual indicator showing that the second rear locking mechanism is unlocked.

There is preferably at least an unlocking of at least one second locking mechanism which is automatically and mechanically driven by pressure on at least part of the third wheels being above a given threshold corresponding to the wheelchair loaded on at least part of the second wheels rather than on all the first wheels, this pressure above a given threshold automatically and mechanically triggering at least one lock visual indicator showing that this second locking mechanism is unlocked.

Hence, thanks to this specific double cascading locking system, the locking of the retractable first wheels can practically no more be activated by accident without wanting to do so, even by a distracted helping walking person, and moreover this locking can indeed practically only be activated when there is a step to climb and when the wheelchair is just adjacent to the step to climb.

Preferably, unlocking of the first front locking mechanism is only possible when the second front locking mechanism is unlocked, and unlocking of the first front locking mechanism is mechanically driven by manual pressure on a first unlock pedal by a user's foot, unlocking of the first rear locking mechanism is only possible when the second rear locking mechanism is unlocked, and unlocking of the first rear locking mechanism is mechanically driven by manual pressure on a second unlock pedal by a user's foot.

There is preferably at least one unlocking of at least one first locking mechanism which is only possible when at least one second locking mechanism is unlocked, and unlocking of at least one first locking mechanism is mechanically driven by manual pressure on at least an unlock pedal by a user's foot.

Hence, thanks to this specific double cascading locking system, the locking of the retractable first wheels can practically no more be activated by accident without wanting to do so, even by a distracted helping walking person, and moreover this locking can indeed practically only be activated when there is a step to climb and when the wheelchair is just adjacent to the step to climb.

Preferably, the first wheels comprise: a pair of first front wheels and a pair of first rear wheels, the second wheels comprise: a pair of second front wheels and a pair of second rear wheels, the pair of first front wheels being paired with the pair of second front wheels, so as to take the load of the wheelchair when the pair of first front wheels is retracted, the pair of first rear wheels being paired with the pair of second rear wheels, so as to take the load of the wheelchair when the pair of first rear wheels is retracted.

Hence, there is a clear coupling, pair by pair, of the first wheels with the second wheels, so that the second wheels help the first wheels when climbing the step, but only help the first wheels to which they are associated.

Preferably, from front to rear of the wheelchair, there are successively: the pair of first front wheels, the pair of second front wheels, the pair of first rear wheels, the pair of second rear wheels, the retraction of the pair of first front wheels is not simultaneous to the retraction of the pair of first rear wheels.

Hence, the smooth transfer from the first lower height of the ground level to the second higher height (second higher height being higher than first lower height) of the step level can be performed step by step, either first front part of the wheelchair and second rear part of the wheelchair, or first rear part of the wheelchair and second front part of the wheelchair.

Preferably, the rotation axis of the second wheels are secured to the structure of the wheelchair so as to remain immobile during retraction of the first wheels.

The rotation axis of each pair of second wheels is offset from the rotation axis of the pair of first wheels to which it is paired, by a gap G which is dependent both on the height H of the stair to be climbed by the wheelchair and on the difference between the diameter D1 of a first wheel and of the diameter D2 of a second wheel.

The relationship is preferably: G=H−(D1−D2)/2

Hence, compromise between structure simplicity and mechanism efficiency is improved.

Preferably, the retracting mechanism of the first wheels rotates the first wheels around a rotation axis offset from the rotation axis of the first wheels.

Hence, retraction of first wheels does not hinder working and rolling of corresponding second wheels.

Preferably, the retracting mechanism comprises return springs which will put back in their original non retracted positions the first wheels when no more retracted, the first locking mechanism then automatically locking the first wheels in their original non retracted positions.

Hence, security of the retracting mechanism is enhanced.

Preferably, the second wheels are rollers, which have smaller diameters than the diameters of the first wheels, and whose circular cross-section area is at most half of the circular cross-section area of the first wheels.

Hence, compromise between structure simplicity and mechanism efficiency is improved. The rollers may be notably smaller than the wheels, since they are supposed to be used only in specific situations: climbing and remaining on specific step, like existing step within a radiology apparatus for instance.

Preferably, the second wheels are not retractable.

Hence, compromise between structure simplicity and mechanism efficiency is improved, since second wheels of limited size, when correctly located, do not need to be retracted, even when the wheelchair drives on the ground, outside the radiological apparatus.

Preferably, the wheelchair also comprises: a base plate supporting the seat, the first wheels being retractable inside this base plate, the second wheels being partly at the height of this base plate and partly below this base plate.

Hence, the encumbrance and the global volume of the wheelchair are further reduced.

Preferably, the first wheels are retracted by moving with respect to this base plate, the second wheels remain immobile with respect this base plate during retractation of the first wheels.

Hence, compromise between structure simplicity and mechanism efficiency is improved.

Preferably, the wheelchair also comprises: the horizontal rotation axis of all the second wheels are fixed with respect to this base plate, the horizontal rotation axis of all first wheels is movable with respect to this base plate during retraction of first wheels, except during retraction of first wheels, the horizontal rotation axis of some first wheels are fixed with respect to this base plate, except during retraction of first wheels the horizontal rotation axis of some other first wheels are vertically orientable with respect to this base plate.

Hence, the maneuverability of the wheelchair is further improved.

Preferably, the wheelchair also comprises: an elevating system: located between the seat and the base plate, elevating the seat with respect to the base plate.

Hence, the wheelchair is further adaptable to different sizes of sitting persons in it.

Preferably, the seat is radiolucent.

Hence, this wheelchair is more easily usable within a radiology apparatus.

Preferably, it is a non-ambulatory patient in functional sitting position wheelchair.

Hence, this wheelchair can be used more easily in medical environment.

Preferably, the altitude difference between the first wheels and the second wheels is within the range 5-20 cm, or within the range 8-12 cm, or about 10 cm. More precisely, it is the altitude difference between the lowest part of the periphery of the first wheels (the part which would rest on the ground and roll on the ground) and the lowest part of the periphery of the second wheels (the part which would rest on the ground and roll on the ground) which is within the range 5-20 cm, or within the range 8-12 cm, or about 10 cm.

Hence, the global encumbrance and the global volume of the wheelchair is further reduced, while at the same time, the height of the step that this wheelchair can climb easily is still notable.

Preferably, the wheelchair is fully mechanical and passive, therefore it comprises: no motor, no electrical supply.

Hence, compromise between structure simplicity and mechanism efficiency is further improved.

Preferably, the wheelchair also comprises: a brake preventing the wheelchair to roll, this brake being activated by manual pressure by a user's foot on a first brake pedal, thereby preferably automatically and mechanically triggering a brake visual indicator showing that this brake is activated, this brake being released by manual pressure by a user's foot on a second brake pedal.

Hence, security of this wheelchair is further improved.

Preferably, the wheelchair also comprises: two rods, whose length is within the range 50 cm-150 cm, preferably about 80 cm, which can be locked in vertical position, which can be unlocked and can be tilted at an angle within the range 30-60 degrees with respect to verticality, preferably about 50 degrees, having handles at their respective distal ends.

Hence, the handiness and the maneuverability of the wheelchair are further improved.

Preferably, the radiological system also comprises: a first security device stopping said vertical scanning, when it detects a patient body part going outside a first predetermined area, so as to avoid collision between said patient body part and said vertically sliding radiation sources covers, and a second security device stopping said vertical scanning, when it detects presence of an object or of a person external to said radiological apparatus within a second predetermined area, so as to avoid collision between said object or person and said vertically sliding radiation sources covers.

Hence, the security of a disabled or non-ambulatory patient sitting in the wheelchair is further improved.

Preferably, in a square array having three rows from A to C and three columns from 1 to 3: said L shaped gantry cover top view recovers squares C1, C2, C3, B3, A3, said 2 radiation sources covers are respectively located within squares B1 and A2, said patient base plate recovers square B2, square A1 remains entirely free and void.

Hence, the maneuverability of the wheelchair is further improved, while the compromise between structure simplicity and mechanism efficiency for the whole radiological system, encompassing both the wheelchair and the radiological apparatus, is further improved.

This second object of the invention is achieved with a mobile patient support comprising: a base having a top side, a bottom side, an anterior portion, and a posterior portion; a seat configured to receive a patient, the seat secured to the top side of the base; a first strut having a proximal end and a distal end, wherein the proximal end is pivotally attached to the bottom side of the base; a first wheel secured near the distal end of the first strut; a second wheel secured to the bottom side of the base posteriorly to the first wheel; wherein the first strut is pivotable between an extended position in which the first wheel is positioned further away from the bottom side of the base portion than the second wheel and a retracted position in which the first wheel is positioned closer to the bottom side of the base portion than the second wheel.

Preferred embodiments comprise one or more of the following features, which can be taken separately or together, either in partial combination or in full combination, with this second object of the invention.

Preferably, the mobile patient support further comprises a first locking mechanism configurable between (1) a locked position in which the first locking mechanism maintains the first strut in the extended position and (b) an unlocked position in which the first locking mechanism retracts the first strut moving the first wheel toward the bottom side of the base.

Preferably, the mobile patient support further comprises a second locking mechanism configurable between (1) a first position in which the second locking mechanism maintains the first locking mechanism in its locked position and (2) a second position in which the first locking mechanism is permitted to transition from the locked position to the unlocked position.

Preferably, the mobile patient support further comprises a visual indicator mechanically connected to the second locking mechanism and configured to provide an indication of whether the second locking mechanism is in the first position or the second position.

Preferably, the second locking mechanism is configured to transition from the first position to the second position when the third wheel is subjected to a sufficient force.

Preferably, the second locking mechanism further comprises a third wheel.

Preferably, the third wheel is positioned relative to the base so as to have a distal-most edge of the wheel extend further from the bottom side of the base than a distal-most edge of the second wheel.

Preferably, the first locking mechanism is configured to be transitioned from the locked position to the unlocked position by a user's foot that applied a pressure to the first locking mechanism.

Preferably, the first wheel is a diameter that is greater than a diameter of the second wheel.

Preferably, the patient support is configured to be transferred onto a raised platform having a height that is greater than the vertical distance between the lowest points of a bottom edge of each of the first and second wheels.

Preferred embodiments of any of former first and second objects of the invention, comprise one or more of the preceding features, which can be taken separately or together, either in partial combination or in full combination, either with the first object of the invention or with the second object of the invention or with both objects of the invention when combined altogether either partially or fully.

Further features and advantages of the invention will appear from the following description of embodiments of the invention, given as non-limiting examples, with reference to the accompanying drawings listed hereunder.

### BRIEF DESCRIPTION OF THE DRAWINGS

FIG. 1 shows a perspective view of an example of a wheelchair according to an embodiment of the invention.

FIG. 2 shows a side view of an example of a wheelchair according to an embodiment of the invention.

FIG. 3 shows a side view of an example of a low part of a wheelchair according to an embodiment of the invention.

FIG. 4 shows a perspective view of an example of a low part of a wheelchair according to an embodiment of the invention.

FIG. 5 shows a side view of an example of a low part of a wheelchair according to an embodiment of the invention, in a first phase to climb a step.

FIG. 6 shows a side view of an example of a low part of a wheelchair according to an embodiment of the invention, in a second phase to climb a step.

FIG. 7 shows a side view of an example of a low part of a wheelchair according to an embodiment of the invention, in a third phase to climb a step.

FIG. 8 shows a side view of an example of a low part of a wheelchair according to an embodiment of the invention, in a fourth phase to climb a step.

FIG. 9 shows a side view of an example of a wheelchair according to an embodiment of the invention, after having climbed a step.

FIG. 10 shows a perspective view of an example of a low part of a wheelchair according to an embodiment of the invention, showing part of the locking system.

FIG. 11 shows a top view of an example of a low part of a wheelchair according to an embodiment of the invention, showing part of the locking system.

FIG. 12 shows a perspective view of an example of a low part of a wheelchair according to an embodiment of the invention, showing part of the locking system.

FIG. 13 shows a perspective view of an example of a skeleton of a wheelchair according to an embodiment of the invention.

FIG. 14 shows a side view of an example of a skeleton of a wheelchair according to an embodiment of the invention, after having climbed a step.

FIG. 15 shows a perspective view of an example of a skeleton of a wheelchair according to an embodiment of the invention, on the ground.

FIG. 16 shows a perspective view of an example of a skeleton of a wheelchair according to an embodiment of the invention, partly on a step.

FIG. 17 shows a perspective view of an example of a skeleton of a wheelchair according to an embodiment of the invention, fully on a step.

### DETAILED DESCRIPTION

On all figures, there are:
A longitudinal direction X, corresponding to a direction front-rear with respect to the wheelchair, in the horizontal plan,
A transversal direction Y, orthogonal to longitudinal direction X, in the horizontal plan,
A vertical direction Z, orthogonal to both longitudinal direction X and transversal direction Y, orthogonal to the horizontal plan.

On all figures, the second wheels 2 will be indifferently mentioned as second wheels 2 or as rollers 2.

FIG. 1 shows a perspective view of an example of a wheelchair according to an embodiment of the invention.

The wheelchair comprises a seat 5 supported by an elevating system 50 which can move the seat 5 vertically and maintain the seat 5 at different heights. The elevating system 50 is supported by a roughly horizontal base plate 3. First wheels 1, comprising two front wheels 11 and two rear wheels 12, are mounted under the base plate 3.

The elevating system 50 is located between the seat 5 and the base plate 3, elevating the seat 5 with respect to the base plate 3. The seat 5 is radiolucent. The wheelchair is a non-ambulatory patient in functional sitting position wheelchair. The wheelchair is fully mechanical and passive, therefore it comprises neither motor, nor electrical supply.

The 2 vertical rods 51, which free hands are handles 52, are extending upward from the horizontal base plate 3. The handles 52 comprise respectively actuators so as to pivot the rods 51 around transversal axis Y. The 2 rods 51 have a length which is within the range 50 cm-150 cm, preferably about 80 cm, and they can be locked in vertical position, and they also can be unlocked and can be tilted at an angle within the range 30-60 degrees with respect to verticality, preferably about 50 degrees, having handles 52 at their respective distal ends.

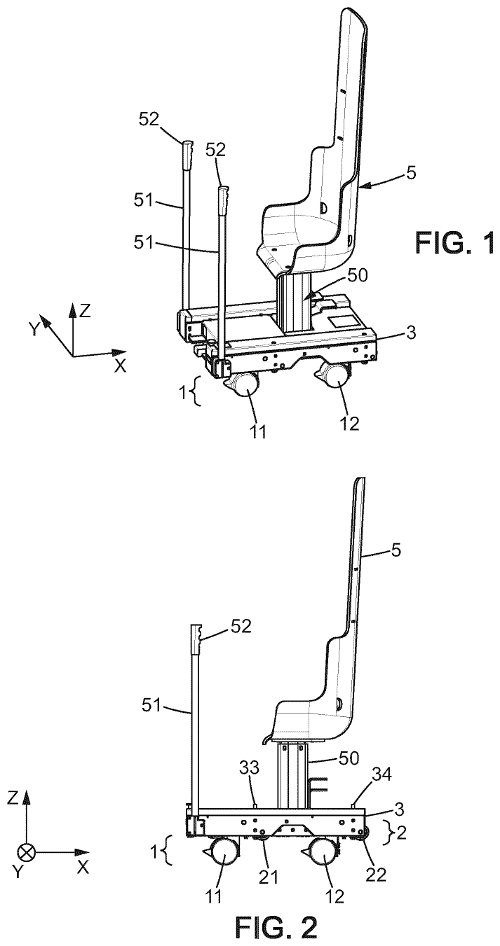

FIG. 2 shows a side view of an example of a wheelchair according to an embodiment of the invention.

Besides the seat 5 and the first wheels 1, the wheelchair also comprises second wheels 2 higher than the first wheels 1. The first wheels 1 are retractable so as to be higher than the second wheels 2, when retracted. The wheelchair indeed comprises two first front wheels 11 associated to two second front wheels 21, and two first rear wheels 12 associated to two second rear wheels 22.

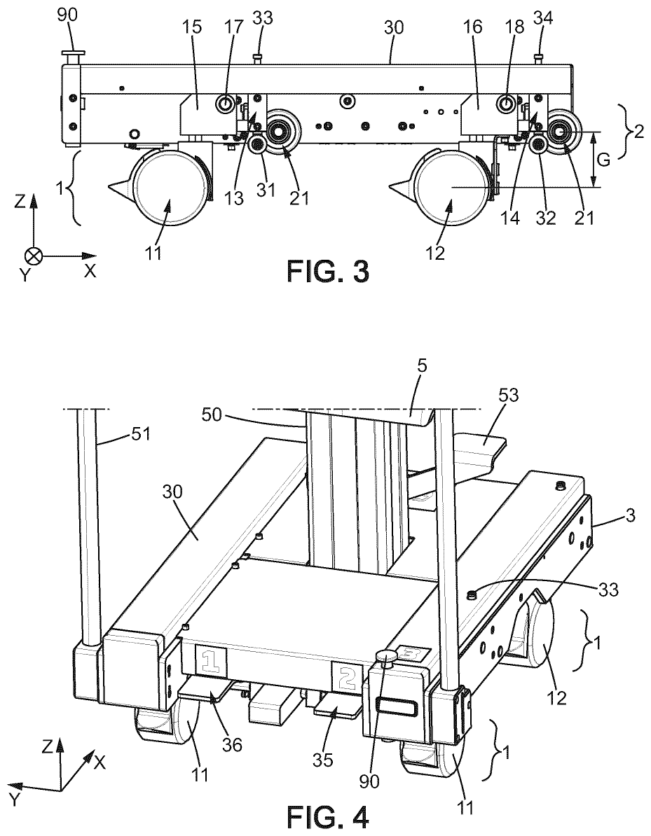

FIG. 3 shows a side view of an example of a low part of a wheelchair according to an embodiment of the invention.

The wheelchair also comprises a first front locking mechanism 13 securing the positions of the first front wheels 11 when not retracted, and a first rear locking mechanism 14 securing the positions of the first rear wheels 12 when not retracted.

The second wheels 2 are rollers 2, which have smaller diameters than the diameters of the first wheels 1, and whose circular cross-section area is at most half of the circular cross-section area of the first wheels 1. The circular cross-section area of the first wheels 1 is the circular surface of the first wheels 1 than can be seen in the plan XZ which is the plan of the FIG. 3. The circular cross-section area of the rollers 2 is the circular surface of the rollers 2 than can be seen in the plan XZ which is the plan of the FIG. 3. The rollers 2 are not retractable, their rotation axis are fixed and immobile with respect to the base plate 3. The altitude difference h between the first wheels 1 and the second wheels 2 is within the range 5-20 cm, or within the range 8-12 cm, or about 10 cm.

Small wheel 31, respectively small wheel 32, have much smaller diameters than the diameters of the rollers 21, respectively rollers 22, less than half their diameters, to which they are respectively associated. Small wheel 31, respectively small wheel 32, are associated to rollers 21, respectively rollers 22, so that, when the wheelchair is loaded on the rollers 21, respectively on the rollers 22, the wheelchair is also pressing on small wheel 31, respectively on small wheel 32, so as to trigger unlocking of the second lock mechanism and so that the visual indicator 33, respectively the visual indicator 34, protrude from upper surface 30 of the base plate 3, mechanically actuated by the small wheel 31, respectively by the small wheel 32, whereas, when the wheelchair is loaded on first wheels 1, then this visual indicator 33, respectively this visual indicator 34, are mechanically retracted within the base plate 3.

FIG. 4 shows a perspective view of an example of a low part of a wheelchair according to an embodiment of the invention.

The wheelchair also comprises a brake preventing the wheelchair to roll, this brake being activated by manual pressure by a user's foot on a first brake pedal, thereby preferably automatically and mechanically triggering a brake visual indicator 90 showing that this brake is activated, this brake being released by manual pressure by a user's foot on a second brake pedal. The brake visual indicators 90 protrudes from upper surface 30 of the base plate 3, mechanically actuated by the first brake pedal being pressed on by user's foot. This brake visual indicators 90 is retracted within the base plate 3, mechanically actuated by the second brake pedal being pressed on by user's foot. The elevating system 50 is mechanically actuated by user's foot pressing on an elevating pedal 53.

The base plate 3 also comprises a first unlock pedal 36 and a second unlock pedal 35. The way these first and second unlock pedals 36 and 35 works will be explained below, with respect to next figures.

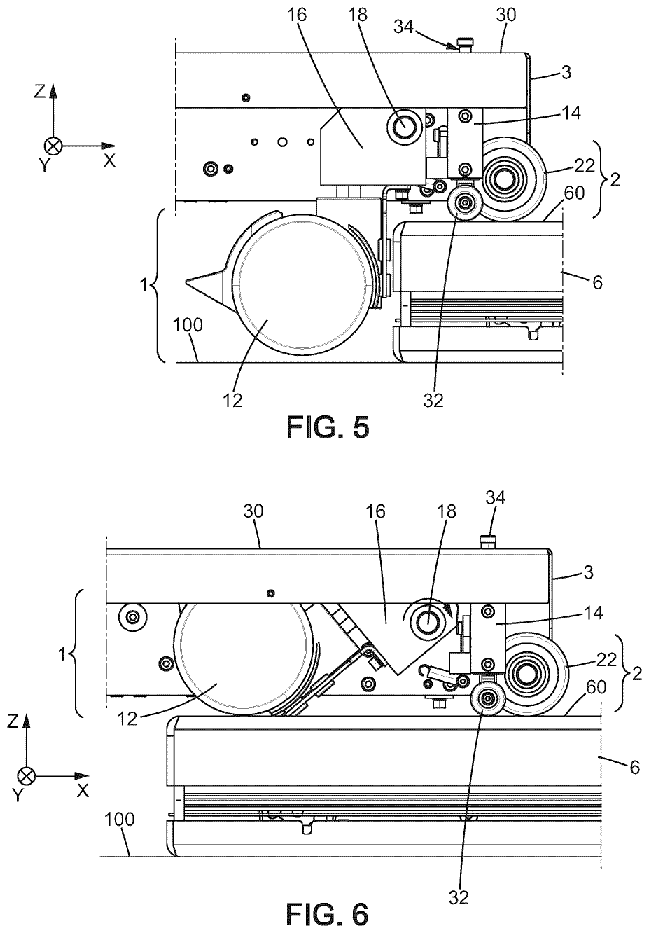

FIG. 5 shows a side view of an example of a low part of a wheelchair according to an embodiment of the invention, in a first phase to climb a step.

There is a step which is a platform 6 resting on the ground floor 100. This platform 6 is an elevating platform which can elevates its upper surface 60 at different heights so that different parts of patient body can be imaged. This platform 6 with an upper surface 60 has been partly climbed up by the wheelchair. The rear rollers 22 are pressing on the upper surface 60 of the platform 6 thereby taking the rear load of the wheelchair, so that the first rear wheels 12 are above the ground floor 100 no more touching this ground floor 100 and then no more taking the rear load of the wheelchair. After the rear locking mechanism 14 of the rear wheels 12 has been unlocked by the load of the wheelchair pressing on the third rear small wheel 32, these rear small wheel 32 also press on the upper surface 60 of the platform 6 thereby mechanically protruding rear visual indicator 34 upward upper surface 30 of base plate 3.

FIG. 6 shows a side view of an example of a low part of a wheelchair according to an embodiment of the invention, in a second phase to climb a step.

This platform 6 with an upper surface 60 has been partly climbed up by the wheelchair. The rollers 22 are pressing on the upper surface 60 of the platform 6 thereby taking the rear load of the wheelchair, and have continued to roll on the upper surface 60 of the platform 6 so that the first rear wheels 12, having abutted against the platform 6, have pivoted around a rear retraction axis 18 with their rear base 16, so as to be retracted within the base plate 3, mechanically actuated by the simple pressure of the platform 6 against the first rear wheels 12. At the end of this second phase, the front load of the wheelchair is still on the first front wheels 11.

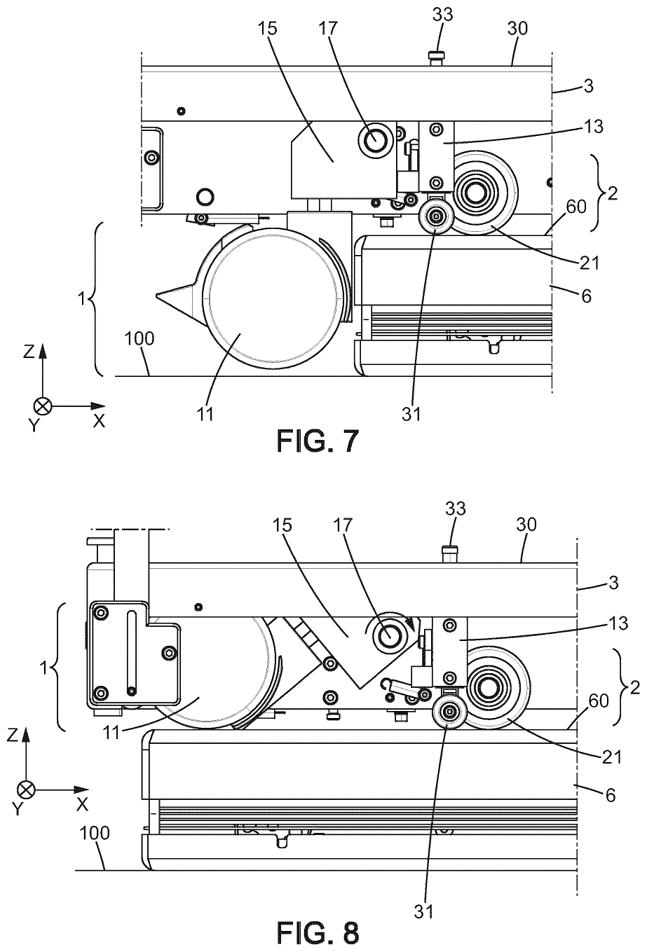

FIG. 7 shows a side view of an example of a low part of a wheelchair according to an embodiment of the invention, in a third phase to climb a step.

This platform 6 with an upper surface 60 has been partly climbed up by the wheelchair. The front rollers 21 are pressing on the upper surface 60 of the platform 6 thereby taking the front load of the wheelchair, so that the first front wheels 11 are above the ground floor 100 no more touching this ground floor 100 and then no more taking the front load of the wheelchair. After the front locking mechanism 13 of the front wheels 11 has been unlocked by the load of the wheelchair pressing on the third front small wheel 31, these front small wheel 31 also press on the upper surface 60 of the platform 6 thereby mechanically protruding front visual indicator 33 upward upper surface 30 of base plate 3.

FIG. 8 shows a side view of an example of a low part of a wheelchair according to an embodiment of the invention, in a fourth phase to climb a step.

This platform 6 with an upper surface 60 has been partly climbed up by the wheelchair. The front rollers 21 are pressing on the upper surface 60 of the platform 6 thereby taking the front load of the wheelchair, and have continued to roll on the upper surface 60 of the platform 6 so that the first front wheels 11, having abutted against the platform 6, have pivoted around a front retraction axis 17 with their front base 15, so as to be retracted within the base plate 3, mechanically actuated by the simple pressure of the platform 6 against the first front wheels 11. At the end of this fourth phase, the whole load of the wheelchair, front load of the wheelchair and rear load of the wheelchair, is no more on any of the first wheels 1, that is to say neither on the first front wheels 11 nor on the first rear wheels 12, but is on the rollers 2, that is to say both on the front rollers 21 and on the rear rollers 22.

The first wheels 1 comprise: a pair of first front wheels 11 and a pair of first rear wheels 12. The second wheels 2 which are the rollers 2 comprise: a pair of second front wheels 21 and a pair of second rear wheels 22. The pair of first front wheels 11 is paired with the pair of second front wheels 21, so as to take the front load of the wheelchair when the pair of first front wheels 11 is retracted within the base plate 3. The pair of first rear wheels 12 is paired with the pair of second rear wheels 22, so as to take the rear load of the wheelchair when the pair of first rear wheels 12 is retracted within the base plate 3.

Since, from front to rear of the wheelchair, there are successively: the pair of first front wheels 11, the pair of second front wheels 21, the pair of first rear wheels 12, the pair of second rear wheels 22, the retraction of the pair of first front wheels 11 is not simultaneous to the retraction of the pair of first rear wheels 12.

The rotation axis of the second wheels 2 are secured to the structure base plate 3 of the wheelchair so as to remain immobile during retraction of the first wheels 1. The retracting mechanism of the first wheels 1 rotates the first wheels 1 around a rotation axis 18 for the first rear wheels 12 or around a rotation axis 17 for the first front wheels 11, which is offset from the rotation axis of the first wheels 1.

The retracting mechanism comprises return springs (not shown) which will put back in their original non retracted positions the first wheels 1 when no more retracted within the base plate 3, the rear first locking mechanism 14 for the first rear wheels 12 and the front first locking mechanism 13 for the first front wheels 11, then automatically locking the first wheels 1 in their original non retracted positions, one after the other.

The base plate 3 supporting the seat 5, the first wheels 1 are retractable inside this base plate 3, more precisely inside the housing of the base plate 3, whereas the second wheels 2 are partly at the height of this base plate 3 and partly below this base plate 3. The first wheels 1 are retracted by moving with respect to this base plate 3, whereas the second wheels 2 remain immobile with respect this base plate 3 during retractation of the first wheels 1. The base plate 3 of the wheelchair also comprises the horizontal rotation axis of all the second wheels 2 which are fixed with respect to this base plate 3, and the horizontal rotation axis of all first wheels 1 which is movable with respect to this base plate 3 during retraction of first wheels 1. Except during retraction of first rear wheels 12, the horizontal rotation axis of first rear wheels 12 are fixed with respect to this base plate 3. Except during retraction of first front wheels 11 the horizontal rotation axis of first front wheels 11 are vertically orientable with respect to this base plate 3.

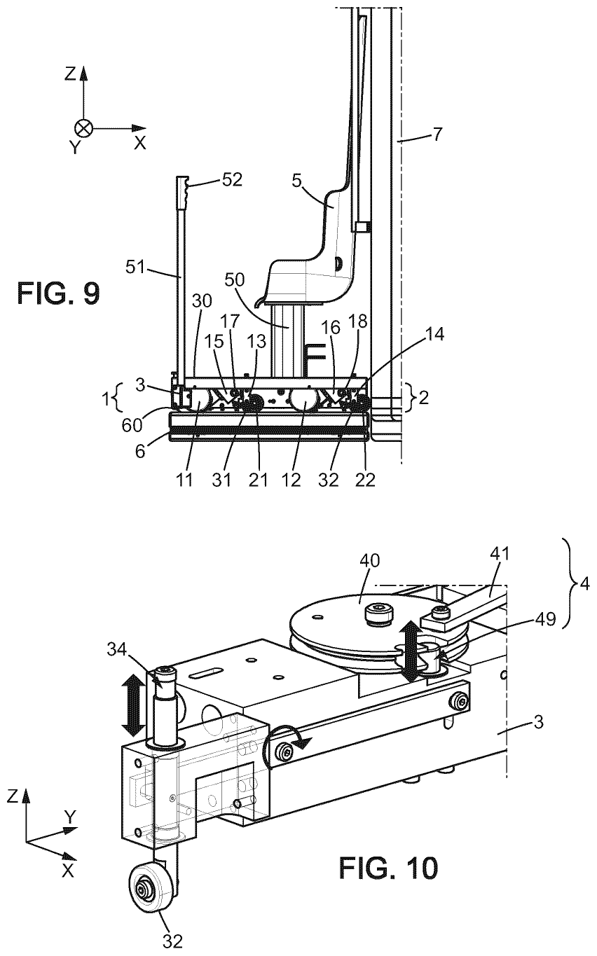

FIG. 9 shows a side view of an example of a wheelchair according to an embodiment of the invention, after having climbed a step.

The whole wheelchair is located on the upper surface 60 of the platform 6 it has climbed. This wheelchair is fully loaded on the rollers 2 which are the second wheels 2, both on the front rollers 21 and on the rear rollers 22, whereas all the first wheels 1, both first front wheels 11 and first rear wheels 12, are retracted within the base plate 3, therefore supporting no more the load of the wheelchair. Base plate 3 is practically abutting against vertical wall 7 of the radiograph apparatus, with the seat 5 leaning against this vertical wall 7 for more stability, so that images are taken with as less as possible moves of the seat 5 with respect to the radiological apparatus.

FIG. 10 shows a perspective view of an example of a low part of a wheelchair according to an embodiment of the invention, showing part of the locking system.

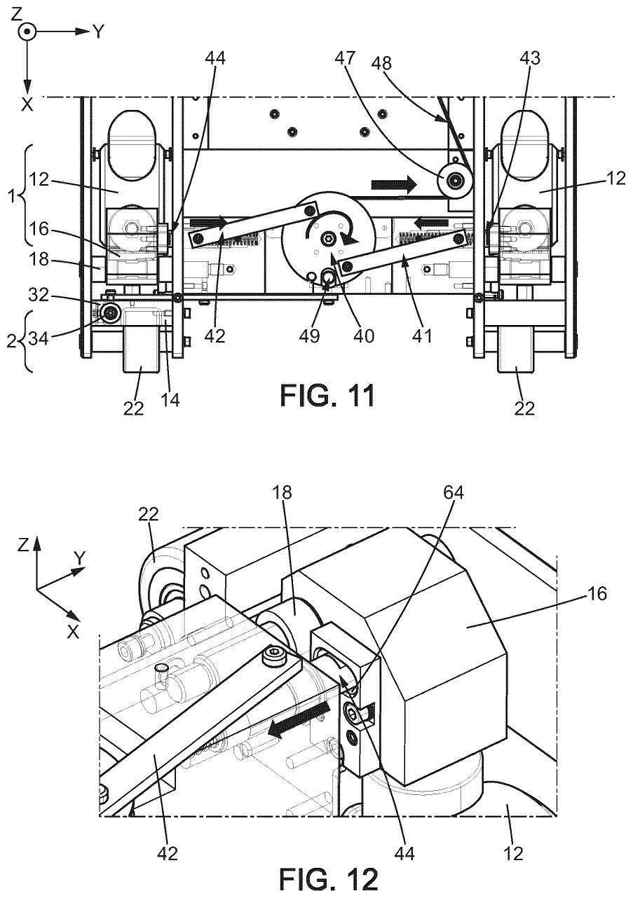

FIG. 11 shows a top view of an example of a low part of a wheelchair according to an embodiment of the invention, showing part of the locking system.

The wheelchair also comprises a second front locking mechanism securing the locking of the first front locking mechanism, a second rear locking mechanism securing the locking of the first rear locking mechanism, both second front locking mechanism and second rear locking mechanism being similar to each other.

Here, the second rear locking mechanism 14 securing the locking of the first rear locking mechanism 4 is shown. Integrated within the base plate 3, there is a locking pin 49 capable of blocking or of releasing a rotating disk 40 which itself pushes or pulls, depending on its rotating position, 2 rods 41 and 42, respectively blocking or releasing security pins 43 and 44, respectively blocking or releasing the first rear wheels 12.

When the locking pin 49 is up (see FIG. 10), it blocks the rotation of the rotating disk 40, thereby maintaining immobile the rods 41 and 42 which respectively keep pushing security pins 43 and 44 into rear bases 16, so as to keep in blocked state the first rear wheels 12.

When the locking pin 49 is down (see FIG. 11), it releases the rotation of the rotating disk 40, thereby pulling respectively the rods 41 and 42 which in turn respectively pull security pins 43 and 44 out of the two rear bases 16 which thereby become released, thereby then making possible the retractation of the first rear wheels 12 by rotation around axis 18 of both the first rear wheels 12 and their rear bases 16, once the rollers 22 have taken the load of the wheelchair.

Once the locking pin 49 is down, the rotating disk 40 is rotated by a cable 48 which is pulled by user's foot pressure on pedal 36 (see FIG. 4). The pulled cable can make an angle by rotating around a pulley 47.

Unlocking of the second rear locking mechanism 14 is automatically and mechanically driven by pressure on the third rear wheel 32 being above a given threshold corresponding to the wheelchair loaded on the second rear wheels 22 rather than on the first rear wheels 12. This pressure above a given threshold automatically and mechanically triggers a lock visual indicator 34 showing that the second rear locking mechanism 14 is unlocked. Unlocking of the first rear locking mechanism 4 is only possible when the second rear locking mechanism 14 is unlocked, and unlocking of the first rear locking mechanism 4 is mechanically driven by manual pressure on a first unlock pedal 36 by a user's foot (see FIG. 4).

In a similar way, even if not represented on FIG. 11, unlocking of the second front locking mechanism is automatically and mechanically driven by pressure on the third front wheel 31 being above a given threshold corresponding to the wheelchair loaded on the second front wheels 21 rather than on the first front wheels 11. This pressure above a given threshold automatically and mechanically triggers a lock visual indicator 33 showing that the second front locking mechanism 13 is unlocked. Unlocking of the first front locking mechanism is only possible when the second front locking mechanism 13 is unlocked, and unlocking of the first front locking mechanism is mechanically driven by manual pressure on a second unlock pedal 35 by a user's foot (see FIG. 4).

Both rear and front first locking mechanism 14 and 13 work in a similar way. When the rollers 2 are not on the platform 6, the third small wheels 31 and 32 are not on the platform 6 and they are in low position. They automatically put the locking pin 49 in up position, which prevents the movement of the wheel retracting mechanism. When the rollers 2 and the third small wheels 31 and 32 are on the platform 6, they are pushed up. This pulls the locking pin 49 down, which frees the movement of the wheel retracting mechanism. The transmission between third small rear wheel 32 and locking pin 49 is performed by rotation of an horizontal bar around the rotation axis shown by the rotating arrow on FIG. 10.

Both rear and front wheel retracting mechanism work in a similar way. When activating the foot pedal, either pedal 36 for the rear wheel retracting mechanism or pedal 35 for the front wheel retracting mechanism, this pulls a cable 48, that makes the rotating disk 40 rotate. The connecting rods 41 and 42 pull the two security pins 43 and 44 out of their housing, freeing the retractation of the first wheels 1. The rotation of the rotating disk 40 is only possible if the second locking mechanism 14 has been unlocked first by translating down the locking pin 49.

FIG. 12 shows a perspective view of an example of a low part of a wheelchair according to an embodiment of the invention, showing part of the locking system.

The rod 42 can either push the security pin 44 into a hole 64 of the first rear base 16 or pull this security pin 44 out of this hole 64 of the first rear base 16.

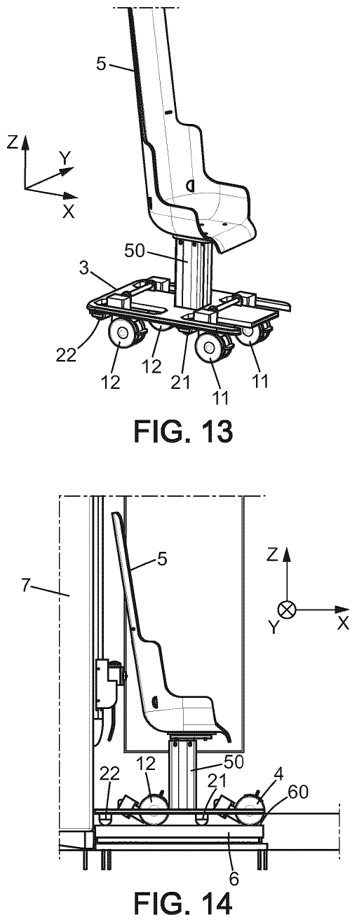

FIG. 13 shows a perspective view of an example of a skeleton of a wheelchair according to an embodiment of the invention.

The wheelchair is on the ground floor and ready to roll on the ground floor on its first wheels 1, that is to say both on its first front wheels 11 and on its first rear wheels 12. The wheelchair is loaded on first wheels 11 and 12, not on second wheels 21 and 22. The first wheels 11 and 12 are in working position, that is to say in extended position, and blocked in this extended position, so as to be able to take the load of the wheelchair.

FIG. 14 shows a side view of an example of a skeleton of a wheelchair according to an embodiment of the invention, after having climbed a step.

The wheelchair is on the upper surface 60 of the platform 6 and either blocked in this position in order to allow for taking of the X-ray images or after such X-ray images having been taken, ready to roll on this upper surface 60 of the platform 6 on its second wheels 2, that is to say both on its second front wheels 21 and on its second rear wheels 22. The wheelchair is loaded on second wheels 21 and 22, not on first wheels 11 and 12. The first wheels 11 and 12 are in retracted position within the housing of the base plate 3 (here the housing of the base plate 3 has been removed to better show inside of the base plate 3), the load of the wheelchair being taken by the second wheels 21 and 22.

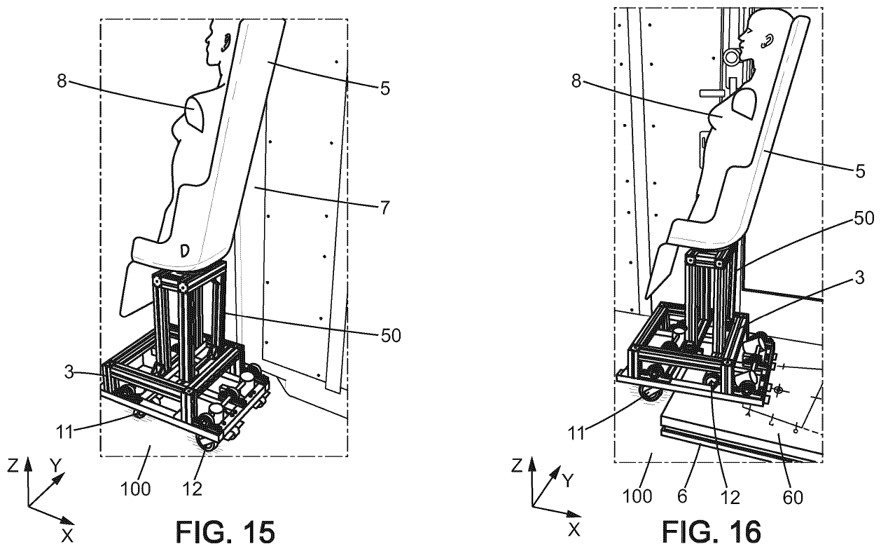

FIG. 15 shows a perspective view of an example of a skeleton of a wheelchair according to an embodiment of the invention, on the ground.

A dummy 8 playing the role of a patient is sitting in the seat 5 of the wheelchair which is rolling on the ground floor 100 and which is entering the X-ray radiology apparatus so as to allow for taking simultaneously both frontal and lateral images of this dummy 8.

FIG. 16 shows a perspective view of an example of a skeleton of a wheelchair according to an embodiment of the invention, partly on a step.

The dummy 8 is still sitting in the seat 5 of the wheelchair which is rolling partly on the ground floor 100 upon first front wheels 11 and partly on the upper surface 60 of the platform 6 upon the second rear wheels 22, the first rear wheels 12 being retracted within the housing of the base plate 3 (here this housing of the base plate 3 has been removed), and which is within the X-ray radiology apparatus so as to allow for taking simultaneously both frontal and lateral images of this dummy 8.

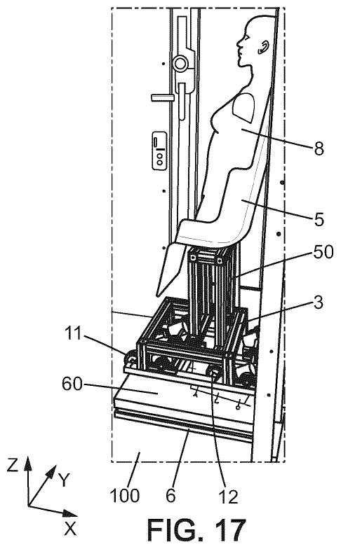

FIG. 17 shows a perspective view of an example of a skeleton of a wheelchair according to an embodiment of the invention, fully on a step.

The dummy 8 is still sitting in the seat 5 of the wheelchair which is rolling on the upper surface 60 of the platform 6 upon both the second front wheels 21 and upon the second rear wheels 22, the first front wheels 11 and the first rear wheels 12 being retracted within the housing of the base plate 3 (here this housing of the base plate 3 has been removed), and which is within the X-ray radiology apparatus, correctly positioned on the platform 6 in the specific position where the frontal and lateral images of this dummy 8 can be taken.

Now a full sequence will be described in relationship with the FIGS. 15-16-17.

The plate base 3 of the wheelchair, which is preferably a medical wheelchair, includes the following mechanisms:
four first wheels 11 and 12, respectively equipped with individual brakes,
a first wheel retractation mechanism to retract the first wheels 11 and 12 of the chair when positioning the wheelchair on the top 60 of the platform 6 of the X-ray imaging apparatus.
a security lock mechanism to lock the position of the wheelchair, once this wheelchair installed on top 60 of the elevating platform 6, as on FIG. 17.

In an initial position, the first wheels 11 and 12 are locked in vertical position by the first wheel locking mechanism. In this initial position, the patient (or here the dummy 8 playing the role of the patient) can be transferred from his or her personal wheelchair into the medical wheelchair according to embodiments of the invention, and this transfer can be easily performed, because it is performed outside the enclosed space of the interior of the cabin of the radiology apparatus. Then, the medical wheelchair with the dummy 8 (or the patient) sitting within the seat 5 of this medical wheelchair, can be moved from the transfer zone to the vicinity of the platform 6, as can be seen on FIG. 15.

The medical wheelchair is pushed towards the platform 6, the rear rollers 22 come in contact with the top 60 of the platform 6. The load is transferred from the first rear wheels 12 to the rear rollers 22; this takes some force, as the first rear wheels 12 are a few millimeters lower than the top 60 of the platform 6, to make sure they are the ones that take the load of the medical wheelchair. The medical wheelchair is pushed until the first rear wheels 12 touch the side 61 made by the step of the platform 6 (see FIG. 16). A visual indicator (34 for the rear rollers 22) shows that the first rear wheels 12 are in the right position. The safety mechanism unlocks the first rear wheels 12 retractation system.

The operator activates the pedal 36 (see FIG. 4), that unlocks the retractation of the first rear wheels 12. This is possible because the first rear wheel locking mechanism 4 has been previously unlocked by the second rear locking mechanism 14. The first rear wheels 12 and their support 16, that is to say their rear base 16, can now rotate around their retractation axis 18 (see FIG. 6).

The medical wheelchair is further pushed toward the platform 6, the front rollers 21 come in contact with the top 60 of the platform 6. The load of the wheelchair is transferred from the first front wheels 11 to the front rollers 21; this takes some force, as the first front wheels 11 are a few millimeters lower than the top 60 of the platform 6, to make sure they are the ones that take the load of the medical wheelchair. The medical wheelchair is pushed until the first front wheels 11 touch the side 61 made by the step of the platform 6 (see FIG. 16).

A visual indicator (33 for the front rollers 21) shows that the first front wheels 11 are in the right position. The safety mechanism unlocks the first front wheels 11 retractation system.

The operator activates the pedal 35 (see FIG. 4), that unlocks the retractation of the first front wheels 11. This is possible because the first front wheel locking mechanism has been previously unlocked. The first front wheels 11 and their support 15 can now rotate around their retractation axis 17 (see FIG. 8).

On FIG. 17, the medical wheelchair is now in scanning position. The X-ray acquisition is performed using the radiology apparatus. After X-ray acquisition has been terminated, the medical wheelchair is pulled down from the platform 6. The first wheels 1 are equipped with return springs, that will place them in vertical position as soon as they are out of the platform 6, successively first the first front wheels 11 and second the first rear wheels 12. Once in vertical position, these first wheels 1 lock automatically in vertical position.

The invention has been described with reference to preferred embodiments. However, many variations are possible within the scope of the invention.

1-36. (canceled)

37. A wheelchair comprising:
a seat,
first wheels,
wherein it also comprises:
second wheels higher than the first wheels,
and wherein:
the first wheels are retractable so as to be higher than the second wheels, when retracted.

38. The wheelchair according to claim 37, further comprising:
two first front wheels associated to two second front wheels,
two first rear wheels associated to two second rear wheels,
a first front locking mechanism securing the positions of the first front wheels when not retracted,
a first rear locking mechanism securing the positions of the first rear wheels when not retracted.
a second front locking mechanism securing the locking of the first front locking mechanism,
a second rear locking mechanism securing the locking of the first rear locking mechanism.

39. The wheelchair according to claim 38, further comprising:
a third front wheel associated to said two first front wheels and to said two second front wheels,
a third rear wheel associated to said two first rear wheels and to said two second rear wheels,
unlocking of the second front locking mechanism is automatically and mechanically driven by pressure on the third front wheel being above a given threshold corresponding to the wheelchair loaded on the second front wheels rather than on the first front wheels,
this pressure above a given threshold automatically and mechanically triggering a lock visual indicator showing that the second front locking mechanism is unlocked,
unlocking of the second rear locking mechanism is automatically and mechanically driven by pressure on the third rear wheel being above a given threshold corresponding to the wheelchair loaded on the second rear wheels rather than on the first rear wheels,
this pressure above a given threshold automatically and mechanically triggering a lock visual indicator showing that the second rear locking mechanism is unlocked.

40. The wheelchair according to claim 38, wherein:
unlocking of the first front locking mechanism is only possible when the second front locking mechanism is unlocked,
and unlocking of the first front locking mechanism is mechanically driven by manual pressure on a first unlock pedal by a user's foot,
unlocking of the first rear locking mechanism is only possible when the second rear locking mechanism is unlocked,
and unlocking of the first rear locking mechanism is mechanically driven by manual pressure on a second unlock pedal by a user's foot.

41. The wheelchair according to claim 37, wherein:
the first wheels comprise:
a pair of first front wheels and a pair of first rear wheels,
the second wheels comprise:
a pair of second front wheels and a pair of second rear wheels,
the pair of first front wheels being paired with the pair of second front wheels, so as to take the load of the wheelchair when the pair of first front wheels is retracted,
the pair of first rear wheels being paired with the pair of second rear wheels, so as to take the load of the wheelchair when the pair of first rear wheels is retracted. wherein
from front to rear of the wheelchair, there are successively:
the pair of first front wheels,
the pair of second front wheels,
the pair of first rear wheels,
the pair of second rear wheels,
the retraction of the pair of first front wheels is not simultaneous to the retraction of the pair of first rear wheels,
and wherein the rotation axis of the second wheels are secured to the structure of the wheelchair so as to remain immobile during retraction of the first wheels.

42. The wheelchair according to claim 37, wherein the retracting mechanism of the first wheels rotates the first wheels around a rotation axis offset from the rotation axis of the first wheels.

43. The wheelchair according to claim 37, wherein:
the retracting mechanism comprises return springs which will put back in their original non retracted positions the first wheels when no more retracted,
the first locking mechanism then automatically locking the first wheels in their original non retracted positions.

44. The wheelchair according to claim 37, wherein the second wheels are rollers, which have smaller diameters than the diameters of the first wheels, and whose circular cross-section area is at most half of the circular cross-section area of the first wheels.

45. The wheelchair according to claim 37, wherein the second wheels are not retractable.

46. The wheelchair according to claim 37, further comprising:
a base plate supporting the seat,
the first wheels being retractable inside this base plate,
the second wheels being partly at the height of this base plate and partly below this base plate.

47. The wheelchair according to claim 46, wherein:
the first wheels are retracted by moving with respect to this base plate,
the second wheels remain immobile with respect this base plate during retractation of the first wheels,
and/or wherein:
the horizontal rotation axis of all the second wheels are fixed with respect to this base plate,
the horizontal rotation axis of all first wheels is movable with respect to this base plate during retraction of first wheels,
except during retraction of first wheels, the horizontal rotation axis of some first wheels are fixed with respect to this base plate,
except during retraction of first wheels, the horizontal rotation axis of some other first wheels are vertically orientable with respect to this base plate,
and/or wherein:
an elevating system:
located between the seat and the base plate,
elevating the seat with respect to the base plate.

48. The wheelchair according to claim 37, wherein the seat is radiolucent, and/or wherein it is a non-ambulatory patient in functional sitting position wheelchair, and/or wherein the altitude difference between the first wheels and the second wheels is within the range 5-20 cm, or within the range 8-12 cm, or about 10 cm, and/or wherein it is:
fully mechanical and passive, therefore it comprises:
no motor,
no electrical supply.

49. The wheelchair according to claim 37, further comprising:
a brake preventing the wheelchair to roll,
this brake being activated by manual pressure by a user's foot on a first brake pedal,
thereby preferably automatically and mechanically triggering a brake visual indicator showing that this brake is activated,
this brake being released by manual pressure by a user's foot on a second brake pedal.

50. The wheelchair according to claim 37, further comprising:
two rods,
whose length is within the range 50 cm-150 cm, preferably about 80 cm,
which can be locked in vertical position,
which can be unlocked and can be tilted at an angle within the range 30-60 degrees with respect to verticality, preferably about 50 degrees,
having handles at their respective distal ends.

51. A radiological system, comprising:
a wheelchair according to claim 37,
and a radiological apparatus comprising:
a gantry encapsulated within a cover,
a patient base plate or platform,
two radiation sources with imaging directions orthogonal to each other, sliding vertically so as to perform vertical scanning of a patient standing on said base plate or platform,
said gantry cover top view being L shaped,
each of said 2 radiation sources:
being located:
outside said L shaped gantry cover,
inside angular sector of said L,
and being encapsulated within a cover sliding vertically with said radiation source it encapsulates.

52. The radiological system according to claim 51, further comprising:
a first security device stopping said vertical scanning, when it detects a patient body part going outside a first predetermined area, so as to avoid collision between said patient body part and said vertically sliding radiation sources covers,
and a second security device stopping said vertical scanning, when it detects presence of an object or of a person external to said radiological apparatus within a second predetermined area, so as to avoid collision between said object or person and said vertically sliding radiation sources covers, wherein:
in a square array having three rows from A to C and three columns from 1 to 3:
said L shaped gantry cover top view recovers squares C1, C2, C3, B3, A3,
said 2 radiation sources covers are respectively located within squares B1 and A2,
said patient base plate or platform recovers square B2,
square A1 remains entirely free and void.

53. A mobile patient support, comprising:
a base having a top side, a bottom side, an anterior portion, and a posterior portion;
a seat configured to receive a patient, the seat secured to the top side of the base;
a first strut having a proximal end and a distal end, wherein the proximal end is pivotally attached to the bottom side of the base;
a first wheel secured near the distal end of the first strut;
a second wheel secured to the bottom side of the base posteriorly to the first wheel;
wherein the first strut is pivotable between an extended position in which the first wheel is positioned further away from the bottom side of the base portion than the second wheel and a retracted position in which the first wheel is positioned closer to the bottom side of the base portion than the second wheel.

54. The mobile patient support of claim 53, further comprising a first locking mechanism configurable between a locked position in which the first locking mechanism maintains the first strut in the extended position and an unlocked position in which the first locking mechanism retracts the first strut moving the first wheel toward the bottom side of the base,
and further comprising a second locking mechanism configurable between a first position in which the second locking mechanism maintains the first locking mechanism in its locked position and a second position in which the first locking mechanism is permitted to transition from the locked position to the unlocked position.
and further comprising a visual indicator mechanically connected to the second locking mechanism and configured to provide an indication of whether the second locking mechanism is in the first position or the second position.

55. The mobile patient support of claim 54, wherein the second locking mechanism further comprises a third wheel,
and wherein the third wheel is positioned relative to the base so as to have a distal-most edge of the wheel extend further from the bottom side of the base than a distal-most edge of the second wheel.

56. The mobile patient support of claim 54, wherein the second locking mechanism is configured to transition from the first position to the second position when the third wheel is subjected to a sufficient force.

57. The mobile patient support of claim 54, wherein the first locking mechanism is configured to be transitioned from the locked position to the unlocked position by a user's foot that applied a pressure to the first locking mechanism.

58. The mobile patient support of claim 53, wherein the first wheel is a diameter that is greater than a diameter of the second wheel.

59. The mobile patient support of claim 53, wherein the patient support is configured to be transferred onto a raised platform having a height that is greater than the vertical distance between the lowest points of a bottom edge of each of the first and second wheels.
